(19) United States
(12) Patent Application Publication
(10) Pub. No.: US 2025/0311922 A1
(43) Pub. Date: Oct. 9, 2025

(54) PREDICTING FUTURE GROWTH OF GEOGRAPHIC ATROPHY USING RETINAL IMAGING DATA

(71) Applicant: Genentech, Inc., South San Francisco, CA (US)

(72) Inventors: Simon Shang GAO, San Francisco, CA (US); Anish Rajesh SALVI, South San Francisco, CA (US); Neha Sutheekshna ANEGONDI, Fremont, CA (US); Julia Gabriella CLUCERU, San Francisco, CA (US)

(21) Appl. No.: 19/244,162

(22) Filed: Jun. 20, 2025

Related U.S. Application Data
(63) Continuation of application No. PCT/US2023/085862, filed on Dec. 22, 2023.
(60) Provisional application No. 63/434,872, filed on Dec. 22, 2022.
(60) Provisional application No. 63/496,202, filed on Apr. 14, 2023.
(60) Provisional application No. 63/519,009, filed on Aug. 11, 2023.

(51) Int. Cl. A61B 3/12; A61B 3/00; A61B 3/10; G06T 7/00; G06T 11/00; G16H 30/40; G16H 50/20

(52) U.S. Cl. CPC A61B 3/12; A61B 3/0025; G06T 7/0016; G06T 11/00; G16H 30/40; G16H 50/20; A61B 3/1025; G06T 2207/20081; G06T 2207/20084; G06T 2207/30041; G06T 2207/30096; G06T 2210/41

(57) ABSTRACT

A method, implemented by one or more computer devices, includes receiving fundus autofluorescence (FAF) image data for a retina of a subject. The FAF image data includes a first FAF image associated with a first point in time. An image input for a deep learning system is generated using the FAF image data. A predicted growth output for a geographic atrophy (GA) lesion in the retina is generated via the deep learning system using the image input. The predicted growth output is associated with at least one future point in time after the first point in time.

### CROSS-REFERENCE TO RELATED APPLICATIONS

This application is a continuation of International Application No. PCT/US2023/085862 filed on Dec. 22, 2023, which claims the benefit of the filing date of, and priority to, U.S. Provisional Patent Application No. 63/519,009, filed on Aug. 11, 2023, U.S. Provisional Patent Application No. 63/496,202, filed on Apr. 14, 2023, and U.S. Provisional Patent Application No. 63/434,872, filed on Dec. 22, 2022, the entire disclosure of each is hereby incorporated herein by reference.

### FIELD

This disclosure is generally directed towards predicting how geography atrophy lesions will change over time and generating a visual depiction of future GA growth locations. More particularly, the present description provides methods and systems for predicting the region of growth (ROG) for geographic atrophy lesion at a future point in time using deep learning.

### BACKGROUND

Age-related macular degeneration (AMD) is a leading cause of vision loss in patients 50 years or older. Geographic atrophy (GA) is a late-stage form of AMD. GA is the degeneration of the retina and can hinder daily activities such as, for example, driving, reading, etc. GA is characterized by progressive and irreversible loss of choriocapillaris, retinal pigment epithelium (RPE), and photoreceptors. GA progression varies between patients and currently, no widely accepted treatment for preventing or slowing down the progression of GA exists. Therefore, evaluating GA progression in individual patients may be important to researching GA and developing an effective treatment. Currently, the diagnosis and monitoring of GA lesion enlargement may be performed using fundus autofluorescence (FAF) images that are obtained by confocal scanning laser ophthalmoscopy (cSLO). This type of imaging technology can be used to measure the change in GA lesions over time. On FAF images, regions of GA can be seen as dark areas and GA progression may be evaluated based on the rate of increase of those dark areas over time.

GA growth rate, which is the change in lesion area over some time period, as measured using FAF images, is widely accepted as an anatomic metric for GA progression in clinical trials. Some currently available techniques for evaluating GA progression using an FAF image, however, may take more time than desired, may be prone to human errors, and/or may product variable results depending on the knowledge and expertise the human graders. For example, some currently available techniques may rely solely on human graders or may be a two-step process in which human graders are required to make manual refinements to software-generated outlines of a GA lesion, the human-refined images then being used to by human graders to determine the GA lesion area and GA growth rate. Thus, the embodiments described herein recognize that it may be desirable to have one or more methods and/or one or more systems that address at least some of the issues described above.

### SUMMARY

In one or more embodiments, a method for predicting the growth of a geographic atrophy (GA) lesion is provided. Fundus autofluorescence (FAF) image data for a retina of a subject may be received. The FAF image data may include a first FAF image associated with a first point in time. An image input for a deep learning system may be generated using the FAF image data. A predicted growth output for a GA lesion in the retina may be generated via the deep learning system using the image input. The predicted growth output may be associated with at least one future point in time after the first point in time.

In some embodiments, the predicted growth output may include a first growth image that illustrates a first predicted region of growth for the GA lesion with respect to the retina of the subject between a first reference point in time and a first future point in time after the first reference point in time. In some embodiments, the predicted growth output may also include a second growth image that illustrates a second predicted region of growth for the GA lesion with respect to the retina of the subject between a second reference point in time and a second future point in time after the second reference point in time. In some embodiments, the second reference point in time and the first reference point in time may be a same point in time or different points in time. In some embodiments, the second future point in time is different from the first future point in time. In some embodiments, the first reference point in time may be the first point in time or a point in time between the first point in time and the first future point in time.

In some embodiments, the predicted growth output may include a growth image that illustrates an area of the retina predicted to be affected by the GA lesion at a selected future point in time. In some embodiments, the predicted growth output may include a computed area for an entire area of the retina in an FAF image of the FAF image data predicted to be affected by the GA lesion at a selected future point in time. In some embodiments, the predicted growth output may include a computed area for an entire area of the retina in an FAF image of the FAF image data predicted to be affected by the GA lesion at a selected future point in time. In some embodiments, the predicted growth output may include a growth image that illustrates an area for new growth of the GA lesion between two points in time. In some embodiments, the predicted growth output may include a computed area for new growth between two points in time.

In some embodiments, the FAF image data may also include a second FAF image associated with a second point in time that is after the first point in time. In some embodiments, generating the image input may include preprocessing each of the first FAF image and the second FAF image such that the image input includes a first preprocessed FAF image and a second preprocessed FAF image. In some embodiments, the predicted growth output may include a growth image illustrating a predicted region of growth for the GA lesion with respect to the retina of the subject with respect to a selected future point in time. In some embodiments, the growth image may include an image background associated with the first FAF image or the second FAF image and a mask over the image background. The mask may identify the predicted region of growth relative to the retina of the subject with respect to the selected future point in time.

In some embodiments, the deep learning system comprises a trained long-short term memory convolutional neural network. In some embodiments, the deep learning system comprises a trained convolutional neural network (CNN). A training dataset used to train the trained CNN may include a plurality of training sets corresponding respectively to a plurality of eyes, where a training set of the plurality training sets may include training FAF images corresponding to four different points in time. In some embodiments, the training FAF images of the training dataset may be stratified by at least one of baseline lesion area, lesion growth rate, foveal involvement, or focality. In some embodiments, the training FAF images of the training set may include four FAF images spaced over time by a consistent time interval.

In one or more embodiments, a method of training a deep learning system is provided. A plurality of training sets for a plurality of retinas of a plurality of subjects may be received. Each training set of the plurality of training sets may include training FAF images for at least two different points in time. A training input for a deep learning system may be generated using the plurality of training sets. The deep learning system may be trained to generate a predicted growth output based on FAF image data for a retina of a selected subject. The predicted growth output may indicate a predicted growth of a GA lesion in the retina with respect to at least one future point in time.

In some embodiments, the predicted growth output may include a growth image illustrating an area of the retina predicted to be affected by the GA lesion with respect to a future point in time. In some embodiments, the predicted growth output may include a computed area for an entire area of the retina predicted to be affected by the GA lesion with respect to a future point in time.

In one or more embodiments, the system may include one or more data processors and a non-transitory computer readable storage medium containing instructions which, when executed on the one or more data processors, cause the one or more data processors to perform any of the methods disclosed herein is provided. In one or more embodiments, a computer-program product tangibly embodied in a non-transitory machine-readable storage medium, including instructions configured to cause one or more data processors to perform any of the methods disclosed herein is provided.

### BRIEF DESCRIPTION OF THE DRAWINGS

For a more complete understanding of the principles disclosed herein, and the advantages thereof, reference is now made to the following descriptions taken in conjunction with the accompanying drawings, in which:

FIG. 1 is a block diagram of a prediction system that includes a deep learning system that receives FAF imagining data and produces growth output, according to an example embodiment.

FIG. 2 is a flowchart diagram of a method of predicting GA lesion growth using a prediction system in accordance with one or more embodiments.

FIG. 3 is a flowchart illustrating an embodiment of a process for training a deep learning system in accordance with one or more embodiments.

FIG. 4A is a schematic diagram of different example deep learning models used to implement the growth prediction system from FIG. 1 in accordance with one or more embodiments.

FIG. 4B is a schematic diagram of different example deep learning models used to implement the growth prediction system 150 from FIG. 1 in accordance with one or more embodiments.

FIG. 5 is an example workflow showing example processing for the three whole lesion models in accordance with one or more embodiments.

FIG. 6 is an example workflow showing example processing for the three multiclass models in accordance with one or more embodiments. For example, FIG. 6 illustrates examples of the input FAF images that may be sent into the whole lesion models (e.g., fourth deep learning model 416, fifth deep learning model 418, sixth deep learning model 420) and the outputs that may be generated as described with respect to FIG. 4B above.

FIG. 7 illustrates example images for an example workflow for post-processing for whole lesion models in accordance with one or more example embodiments.

FIG. 8 illustrates example images for an example workflow for post-processing for multiclass models in accordance with one or more example embodiments.

FIG. 9 illustrates example images for an example workflow for post-processing of multiclass whole lesion models in accordance with one or more example embodiments.

FIG. 10 illustrates examples of the predicted growth images generated for different deep learning models in accordance with one or more embodiments.

FIG. 11 is a table illustrating various results of the example experiment for each of the different six models for the training, validation, and test sets in accordance with one or more embodiments.

FIG. 12 is a block diagram illustrating an example of a computer system in accordance with one or more embodiments.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DETAILED DESCRIPTION

### I. Overview

The ability to accurately predict how geographic atrophy (GA) will progress over time may be useful in many different scenarios. As one example, predictions about GA progression may be used to improve patient stratification in clinical trials where the goal is to slow GA progression, thereby allowing for improved assessment of treatment effects. Additionally, in some cases, predictions about GA progression may be used to understand disease pathogenesis via correlation to genotypic or phenotypic signatures. Further, prediction of GA growth can be used in clinical trials for enrichment, stratification, or covariate adjustment and in clinical practice for patient counseling. In addition to GA growth, the location of GA lesions has an impact on vision. Consequently, predicting the future region of growth of GA lesions may be useful for identifying patients with a higher risk of vision loss.

A GA lesion can be imaged by various imaging modalities. FAF images have been used to quantify the GA lesion area. GA growth rate, which is the change in lesion area over some time period, as measured using FAF images, is widely accepted as an anatomic metric for GA progression in clinical trials. Currently available techniques for evaluating GA progression using an FAF image, however, rely on human graders to first manually identify the portion of an FAF image that is the GA lesion. In some cases, this first step is semi-automated, relying on the human grader to make manual refinements and/or corrections to a software-generated initial outline of the GA area. Then, the identified portion of the FAF image is evaluated to determine the GA lesion area and GA growth rate. These techniques may involve a two-step process that can take more time than is desirable, may be prone to human error, may be less accurate than desired, and/or may produce variable results depending on the knowledge and expertise of one or more human graders. Further, these types of techniques are meant for individual time points and are therefore unable to visually present to a medical professional (e.g., clinician, healthcare provider, etc.) how the FAF image might look in the future (e.g., 3 months, 6 months, 9 months, 1 year, etc. later).

Accordingly, a desire exists for methods and systems that improve the speed, efficiency, and accuracy associated with predicting GA lesion growth and that provide a way of visualizing the region of growth for GA lesions for a future point in time. The present disclosure describes various embodiments for using deep learning to predict future region of growth of geographic atrophy lesions from retinal imaging data such as, for example, FAF images. In present embodiments, GA growth rate (e.g., annualized growth rate) may be predicted from baseline FAF images.

In one or more embodiments, a method is provided for predicting future geographic atrophy expansion based on retinal imaging data. This retinal imaging data may take the form of, for example, FAF imaging data. FAF imaging data of an eye of a subject for a first point in time may be received. A deep learning system processes the FAF imaging data and generates a growth image with a mask that identifies a region of growth for a geographic atrophy lesion with respect to a future point in time. For example, the mask may identify the area that is predicted to be affected by the GA lesion at a future point in time. In some cases, the mask identifies new growth or, in other words, the new growth that is predicted between a reference point in time and the future point in time. In some cases, the deep learning system may also be used to predict the computed area (e.g., mm²) for the region of growth. In other embodiments, the deep learning system may use the FAF imaging data to generate multiple growth images for different future points in time.

The predictions may be generated with an accuracy that can be successfully relied upon for use in clinical practice. For example, the growth image may be used to determine whether a subject is a candidate for a clinical trial, to which clinical trial to assign the subject, how to customize a treatment for the subject, how to monitor the progress of the subject during the clinical trial, or a combination thereof. The techniques described herein can be used to predict the prognosis of one or more subjects, predict the responsiveness of one or more subjects to various treatments, identify the treatment predicted to be effective for an individual subject, assign one or more subjects into an appropriate arm within a clinical trial, or a combination thereof.

The growth image may be used to generate an output that includes an indication of whether a subject is eligible for a clinical trial for testing a medical treatment for geographic atrophy. In some embodiments, this output may be used to enroll the subject in the clinical trial, exclude the subject from participating in the clinical trial, customize a protocol in the clinical trial for the subject, or enroll the subject in a different clinical trial.

### II. Geographic Atrophy (GA) Lesion Prediction

### II.A. Example Prediction System

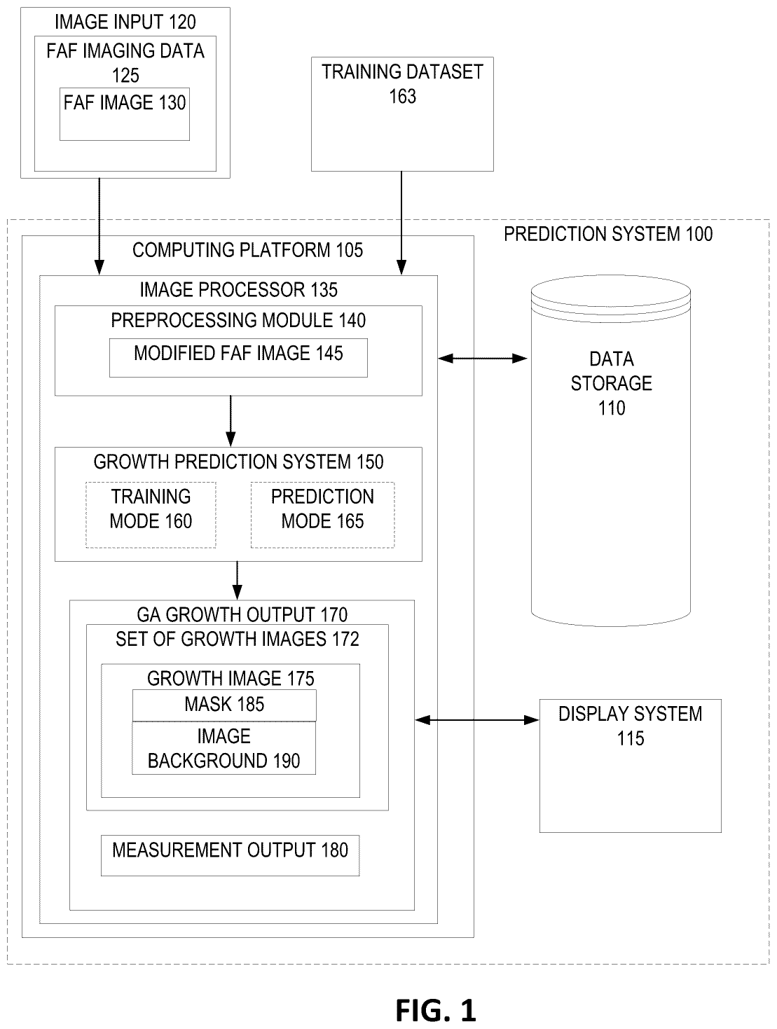

Referring now to the figures, FIG. 1 is a block diagram of a prediction system 100 in accordance with various embodiments. Generally, the prediction system 100 is used to predict the progression of geographic atrophy (GA) lesions in the retinas of subjects. As illustrated, the prediction system 100 includes a computing platform 105, data storage 110, and a display system 115.

The computing platform 105 may take various forms. For example, in one embodiment, the computing platform 105 includes a single computer (or computer system), but in another embodiment the computing platform 105 includes multiple computers in communication with each other. In other examples, the computing platform 105 takes the form of a cloud computing platform. As illustrated, the data storage 110 and the display system 115 are each in communication with the computing platform 105. In some examples, the data storage 110, or the display system 115, or both may be considered part of or otherwise integrated with the computing platform 105. Thus, in some examples, the computing platform 105, the data storage 110, and the display system 115 may be separate components in communication with each other, but in other examples, some combination of these components may be integrated together.

The computing platform 105 of the prediction system 100 is configured to receive or otherwise access image input 120. The image input 120 may include one or more images obtained for one or more subjects. The image input 120 may include retinal imaging data such as, for example, without limitation, FAF imaging data 125. The FAF imaging data 125 includes one or more FAF images, such as FAF image 130, each of which captures a retina of a subject. Generally, the retina of a subject has a geographic atrophy (GA) lesion or is expected to have a GA lesion. This GA lesion may be a continuous or discontinuous region of the retina that has suffered degeneration (e.g., chronic progressive degeneration). The GA lesion may include one lesion (e.g., one continuous lesion region) or multiple lesions (e.g., discontinuous lesion region comprised of multiple, separate lesions).

In one or more embodiments, the FAF imaging data 125 includes one or more reference FAF images that are captured for one or more reference points in time. The one or more reference points in time may include, for example, a baseline point in time, a point in time that is 6 months after a first treatment, a point in time that is 3 months after a first treatment, a point in time that is 12 months after a first treatment, or some other type of reference point in time. In one or more embodiments, the baseline point in time may be a point in time prior to treatment, the same day as a treatment dose (e.g., a first treatment dose), the same day as an initial diagnosis of GA, the same day as a screening conducted for GA, or some other type of baseline or reference point in time. For example, a first FAF image, which may be used as a reference image, may correspond to 0 months (“T1”). A second FAF image may be taken 6 months (“T2”) after the T1 FAF image. A third FAF image may be taken 12 months (“T3”) after the T1 FAF image (6 months after the T2FAF image). A fourth FAF image may be taken 18 months (“T4”) after the T1 FAF image (12 months after the T2 FAF image and 6 months after the T3 FAF image). The FAF image 130 as illustrated in FIG. 1 is one example of a FAF image in FAF imaging data 125. The FAF image 130 corresponds to a reference point in time and captures a GA lesion. While the example interval of time disclosed herein is 6 months, the interval of time may be 1 month, 3 months, 9 months, or measured in weeks. In some embodiments, the FAF images associated with each eye are spaced by a consistent interval of time (e.g., 6 months).

The prediction system 100 includes an image processor 135, which may be implemented using hardware, software, firmware, or a combination thereof. In one or more embodiments, the image processor 135 is implemented in the computing platform 105. The image processor 135 receives the image input 120 for processing. For example, the image input 120 may be sent as input into the image processor 135, retrieved from the data storage 110 or some other type of storage (e.g., cloud storage), or received in some other manner.

In various embodiments and as illustrated in FIG. 1, the image processor 135 includes a preprocessing module 140, which processes the image input 120 (e.g., the FAF imaging data 125) to create a modified FAF image 145 and then sends the modified FAF image 145 to a growth prediction system 150 to generate growth output 170. The preprocessing module 140 is illustrated as a separate component from the growth prediction system 150 in FIG. 1.

However, in some embodiments, the growth prediction system 150 and the preprocessing module 140 may be considered one component. In such embodiments, the growth prediction system 150 may receive the FAF image 130 as input and process the FAF image 130 to generate the modified FAF image 145. The growth prediction system 150 may then generate the growth output 170 for the GA lesion captured in the FAF image 130 in some embodiments. The preprocessing may include scaling, resizing, cropping, horizontal flipping, vertical flipping, normalizing image intensities, adding and/or removing noise, translating, and other such preprocessing operations. The resizing may include resizing the FAF image 130 into a selected pixel size (e.g., 512 pixels by 512 pixels). The normalization of image intensities may include normalizing the intensity values of the pixels in the FAF image 130 to a selected scale (e.g., a scale from 0 to 1, a scale from −1 to 1, or another type of scale).

The growth prediction system 150, which may include a machine learning model (e.g., deep learning model), may be implemented in any of a number of different ways. In one or more embodiments, growth prediction system 150 may be a deep learning system that includes one or more deep learning models. For example, the growth prediction system 150 may include Artificial Neural Networks (ANNs), such as a perceptron, a multilayer perceptron (MLP), an autoencoder (AE), a convolution neural network (CNN), a recurrent neural network (RNN), long short term memory (LSTM), a grated recurrent unit (GRU), a restricted Boltzmann Machine (RBM), a deep belief network (DBN), a bidirectional recurrent deep neural network (BRDNN), a generative adversarial network (GAN), and deep Q-networks, a neural autoregressive distribution estimation (NADE), an adversarial network (AN), attentional models (AM), a spiking neural network (SNN), deep reinforcement learning, or some other model.

In one or more embodiments, the growth prediction system 150 may be implemented using a prediction neural network (NN) system. The prediction NN system may include any number of or combination of neural networks. In one or more embodiments, the prediction NN system takes the form of a convolutional neural network (CNN) that includes one or more neural networks. In some cases, the growth prediction system 150 includes multiple subsystems and/or layers, each including one or more neural networks. In other embodiments, the prediction NN system takes the form of a long-short term memory (LSTM) UNet that includes one or more neural networks. Each of these one or more neural networks may itself be a convolutional neural network.

In other embodiments, the prediction NN system takes the form of a 2-dimensional U-Net CNN. The U-Net may consist of an encoder (contracting path), which converts an image into feature maps, and a decoder (expanding path), which converts the feature maps into a probability map of equal size to the input image. The image may include, for example, the image input 120. The encoder may extract image features of different spatial resolutions, which may in turn be used by the decoder to derive an accurate segmentation mask.

In some embodiments, the encoder is a 34-layer residual neural network (ResNet) backbone to extract features at different resolutions, an encoder depth of 4, and pretrained ImageNet weights, where the encoder depth signifies the number of stages and the feature size decreases with each additional stage. The decoder may use batch normalization between the convolutional and activation layers and may have a depth of 4 with decoder channels of (128, 64, 32, 16).

The growth prediction system 150 may be used in either training mode 160 with a training dataset 163 (also referred to as training input) or prediction mode 165. In the prediction mode 165, the growth prediction system 150 is used to generate the growth output 170. The growth output 170 generated by the growth prediction system 150 may include, for example, a set of growth images 172 (e.g., which includes one or more growth images such as growth image 175) and/or one or more measurement outputs, such as measurement output 180. In one embodiment, the growth image 175 identifies a region of growth for a geographic atrophy lesion with respect to a future point in time.

In one or more embodiments, the growth image 175 may include a mask 185, an image background 190, or both. The image background 190 may be the image layer under the mask 185. The image background 190 may be, for example, the FAF image 130, the modified FAF image 145, or some other representation of the FAF image 130. In one or more embodiments, the image background 190 has dimensions that are equal in size or proportional to (e.g., a same aspect ratio) as the FAF image 130.

Generally, the mask 185 indicates a predicted growth for the geographic atrophy (GA) lesion (which may be continuous or discontinuous) for the future point in time, For example, the mask 185 may be overlaid on the image background 190, which may be the FAF image 130, to identify the portion of the FAF image 130 that corresponds to the predicted growth for the geographic atrophy (GA) lesion for the future point in time. The mask 185 indicates the area that is predicted to be affected by geographic atrophy at the future point in time (e.g., some number of days, weeks, months, or years, after the reference point in time).

In one or more embodiments, the mask 185 takes the form of a boundary or outline. The boundary or outline may be dashed, solid, dotted, or some other variation. The boundary or outline may include a variety of thicknesses. In other embodiments, the mask 185 is an opaque block or other type of graphical feature overlaid over the FAF image 130 that identifies the entire area (or alternatively, a selected percentage of the area) that is predicted to be affected by the GA lesion. The mask 185 can be presented in using any type of grayscale, any color(s), any hashing, any pattern, or the like that can be distinguished from the image background 190. In one or more embodiments, the mask 185 is identified on the pixel level of the FAF image 130. In some embodiments, the mask 185 is identified by the left most or right most pixel of every row in the FAF image 130.

As discussed above, the mask 185 may identify the area or portion of the image background 190 that is predicted to be affected by the GA lesion at the future point in time. When a reference point in time is prior to treatment, the mask 185 may identify the predicted region of growth (ROG) for the GA lesion at the baseline point in time. In other embodiments, the mask 185 identifies the difference between the area of the image background 190 that is predicted to be affected by the GA lesion at the future point in time and the reference point in time. In this manner, the region of growth predicted by the growth prediction system 150 may be the new growth between the reference point in time and the future point in time. In still other examples, the mask 185 includes multiple predictions for multiple points in time after a baseline point in time or other type of reference point in time.

As discussed above, the mask 185 may indicate the predicted region of growth for the GA lesion by identifying the predicted region of growth. In other embodiments, the mask 185 may identify the portions of the FAF image 130 that are not predicted as being the region of growth for the GA lesion to thereby indicate the predicted growth. For example, the mask 185 may be overlaid over all portions of the FAF image 130 that are predicted to not be associated with GA lesion at the future point in time such that any portion of the FAF image 130 not covered by the mask 185 indicate the predicted growth.

In one or more embodiments, the growth prediction system 150 also generates the measurement output 180, which may be a measurement for the region of growth for the GA lesion. This measurement may be, for example, the computed area (e.g., mm²) for the region of growth. The measurement may be the computed area of the entire area predicted to be affected by the GA lesion at the future point in time or the computed area for the new growth between the reference point in time and the future point in time. In some embodiments, the measurement output 180 is presented on the growth image 175. In other embodiments, the measurement output 180 is presented separately from the growth image 175.

As previously noted, the growth prediction system 150 is trained when in the training mode 160. In the training mode 160, the growth prediction system 150 is trained using the training dataset 163. The training dataset 163 includes an FAF image dataset that is selected to ensure the growth prediction system 150 can be used in the prediction mode 165 with the desired level of accuracy. In one or more embodiments, the training dataset 163 includes FAF images obtained via one or more studies (e.g., clinical studies, research studies, etc.). When the FAF images are obtained from multiple studies, the studies are selected such that the inclusion criteria for the studies are the same. Ensuring that the same inclusion criteria were used in the studies helps ensure a certain type of consistency across the FAF images that will improve training accuracy and thereby, prediction accuracy.

### II.B. Example Methodologies for Predicting GA Lesion Growth Using Prediction System

As previously noted with respect to FIG. 1, the growth prediction system 150 of prediction system 100 may be used in training mode 160 or prediction mode 165. Below, example methods for using growth prediction system 150 in these modes are described in further detail.

### II.B.1. Example Methods With Prediction System in Prediction Mode

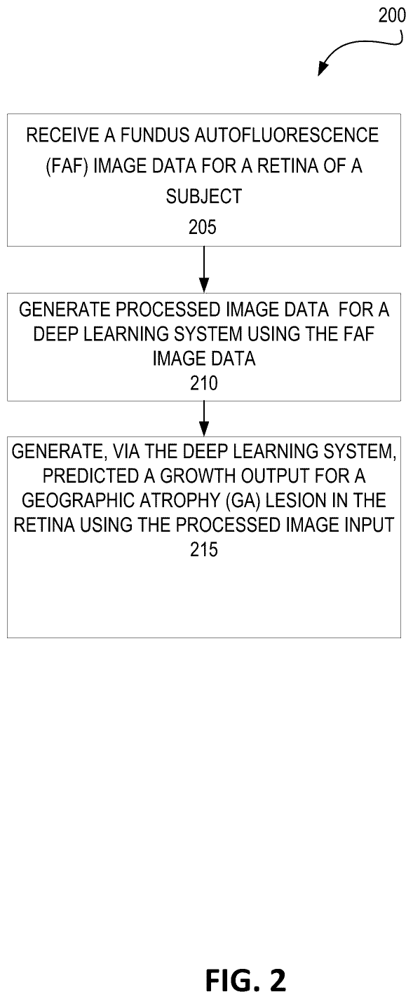

FIG. 2 is a flowchart diagram of a method of predicting GA lesion growth using a prediction system in accordance with one or more embodiments. In FIG. 2, the prediction system used may be prediction system 100 in FIG. 1. Accordingly, process 200 in FIG. 2 is described with continuing reference to FIG. 1 and prediction system 100 of FIG. 1.

Step 205 includes receiving fundus autofluorescence (FAF) image data for a retina of a subject. In some embodiments, and at the step 205, the prediction system 100 receives the FAF image data. In some embodiments, the FAF image data received, or otherwise accessed, is the image input 120. As noted above, the FAF image data 120 may include the FAF imaging data 125, which may include one more FAF images, such as for example the FAF image 130. Generally, one of the FAF images is associated with a first point in time. The first point in time or first reference point in time may be, for example, a baseline point in time (e.g. the time of an initial diagnosis of GA, which may be referred to as time (0) or 0 months, or a point in time after the baseline point in time (e.g., 3 months, 6 months, 9 months, 12 months, 18 months, etc. after baseline). The first future point in time may be 6 months, 1 year, 18 months, 2 years, or some other point in time after the first reference point in time. The first reference point in time may be, for example, the first point in time (e.g., the baseline point in time) or another point in time between a baseline point in time and the future point in time. In some embodiments, the FAF image data 120 received comprises a T1 FAF image or a T2 FAF image, and in other embodiments, the FAF image data received comprises the T1 FAF image and the T2 FAF image.

Step 210 includes generating processed image data for a deep learning system using the FAF image data at step 210. In some embodiments, and at the step 210, the prediction system 100 generates processed image data for the deep learning system using the received FAF imaging data. In some embodiments, the deep learning system is the growth prediction system 150 and the processed image data includes the modified FAF image 145. In some embodiments, generating the modified FAF image 145 for the growth prediction system 150 using the FAF image data 125 includes sending the FAF image data 125 to the preprocessing module 140 or to the growth prediction system 150 for preprocessing as an input. As noted previously, the preprocessing may include scaling, resizing, cropping, horizontal flipping, vertical flipping, normalizing image intensities, adding and/or removing noise, translating, and other such preprocessing operations.

Step 215 includes generating, via the deep learning system, a predicted growth output for a geographic atrophy (GA) lesion in the retina using the processed image data at step 215. In some embodiments, and at the step 215, the prediction system 100 generates, via the growth prediction system 150, a predicted growth output for a geographic atrophy (GA) lesion in the retina using the processed image data. In some embodiments, the predicted growth output comprises the GA growth output 170 and the prediction NN system generates the GA growth output 170.

The growth prediction system 150 may include one or more models from a variety of models. Additional detail is provided with respect to examples below, but the prediction NN system of the growth prediction system 150 may be, for example, a whole lesion model, a Simple UNet model, a multi-channel UNet model, a sequential label UNet model, a LSTM UNet model, or another CNN model. In some embodiments, a whole-lesion model is be used. In other embodiments, the growth prediction system 150 may be trained using the T4 whole-lesion as ground truth. In some cases, the growth prediction system 150 may infer the T4 whole lesion from the T2 FAF image. The growth prediction system 150 may be a Simple U-Net. In some embodiments, the growth prediction system 150 may infer the T4 whole lesion from the combination of T1 and T2 FAF images. The growth prediction system 150 may be a Multichannel U-Net. In some embodiments, the growth prediction system 150 may infer the T3 and T4 whole lesions, respectively, from the T2 FAF image. The growth prediction system 150 may be a Sequential Label U-Net. In still other embodiments, the growth prediction system 150 may infer the T4 whole lesion from the combination of T1 and T2 FAF images. The growth prediction system 150 may be, for example, an LSTM UNet model. In some embodiments, the growth prediction system 150 includes or comprises a multiclass model. The multiclass model(s) may be trained on a multiclass ground truth. In some embodiments, the growth prediction system 150 may infer the classes T2 whole lesion and the 1-year region of growth (ROG) (e.g. the region of growth between the T4 lesion area and the T2 lesion area, i.e. T4−T2 ROG) from the T2 FAF image. The growth prediction system 150 may infer both the T2 whole lesion and 1-year ROG (e.g. T4−T2 ROG) from the combination of T1 and T2 FAF images. The growth prediction system 150 may infer the T2 whole lesion, 6-month ROG (e.g. T4−T3 ROG and T3−T2 ROG) from the T2 FAF image.

The growth prediction system 150 may be a Sequential U-Net. With each of these examples of the growth prediction system 150, the prediction NN system can provide end-to-end prediction in which the input is automatically processed to the predict GA growth output 170. Human intervention is not needed in the prediction mode. Generally, the growth prediction system 150 has been trained using a training dataset that ensures GA lesion growth is predicted with at least a threshold level of accuracy, which may defined based on, for example, a performance metric.

The GA growth output 170 generated by the growth prediction system 150 is associated with at least one future point in time after the first point in time. That is, the GA growth output 170 is associated with a point in time that is chronologically after the first point in time, and is therefore, a future point in time relative to the first point in time. When the FAF image data received comprises a T1 FAF image, then the future point in time may be associated with T2, T3, T4, or later, and when the FAF image data received comprises a T2 FAF image then the future point in time may be associated with T3, T4, or later. The GA growth output 170 indicates a predicted growth of a geographic atrophy (GA) lesion in the retina for at least one future point in time and may include one or more growth images. For example, the GA growth output 170 may include a first growth image and associated first measurement output and a second growth image and associated second measurement output. However, and as noted earlier, the measurement outputs may be excluded in some embodiments. In some embodiments, when the FAF image data 125 received comprises a T2 FAF image at the step 205, then the first growth image and first measurement output are associated with a T3 lesion and the second growth image and the second measurement output are associated with a T4 lesion. However, the combination of growth outputs varies and additional examples are provided below.

In some embodiments, the first growth image and associated first measurement output are associated with a first future point in time and a second growth image and associated second measurement output are associated with a second future point in time. The second future point in time and the first future point in time may be a same point in time or different points in time. As one example, the first future point in time is associated with a predicted GA lesion growth at 6 months from baseline, and the second future point in time is associated with a predicted GA lesion growth at 1 year from baseline.

One example growth measurement 180 includes a computed area (e.g., mm²) for the combined affected area, which includes the area already affected at the first point in time and the predicted growth area for the future point in time. Another measurement may, for example, include the region of growth (e.g., computed area for the predicted growth area for the future point in time) and omit the area already affected at the first point in time. However, in other embodiments, the growth measurement 180 includes the region of growth between two future points in time. For example, and when the growth output comprises predicted growth for a first future period in time and predicted growth for a second, future period in time, then the region of growth may include the difference between the areas associated with the second and first future points in time. In some embodiments, the measurement output 180 is overlaid over or presented alongside a growth image 175. In other embodiments, measurement output 180 is presented separately from the growth image 175. An image may include a photograph, an annotated photograph, a graphical depiction, etc.

Whereas the growth measurement 180 is represented by a unit of measurement such as mm², pixel, etc., the mask 185 may identify predicted growth location(s) relative to the retina of the subject for a selected future point in time. That is, the mask 185 provides a visual indication of which areas of the retina will be affected at the future point(s) in time. In one or more embodiments, the mask 185 may identify the area of the image background 190 that is affected in addition to the area that is to be predicted to be affected by the GA lesion at the future point in time. In other embodiments, the mask 185 identifies the difference between the areas of the image background 190 that is predicted to be affected by the GA lesion at the future point in time and the reference point in time (e.g., identify area of growth predicted after the reference point in time). Still in other embodiments, the mask 185 identifies the difference between the areas of the FAF image background that is predicted to be affected by the GA lesion at two different future points in time.

In one or more embodiments, the step 215 can be repeated to generate a third growth image and/or a third measurement output for a third future point in time. The prediction system 100 is not limited to generating three growth images and/or measurement outputs for three future points in time, and can generate any number of growth images and/or measurement outputs for any number of future points in time.

In one or more embodiments, the GA growth output 170 may be sent to the display system 115 over one or more communication links (e.g., wired, wireless, and/or optical communications links), stored in the data storage 110, or both. The display system 115 includes one or more display devices in communication with computing platform 105. The display system 115 may be separate from or at least partially integrated as part of the computing platform 105.

In some embodiments, the GA growth output 170 is transmitted as a report that may be viewed on the display system 115. The report may include, for example, without limitation, at least one of a table, a spreadsheet, a database, a file, a presentation, an alert, a graph, a chart, one or more graphics, or a combination thereof.

For example, the GA growth output 170 may be used to determine whether a subject is a candidate for a clinical trial, to which clinical trial to assign the subject, how to customize a treatment for the subject, how to monitor the progress of the subject during the clinical trial, or a combination thereof. The method 200 and/or the GA growth output 170 can be used to predict the prognosis of one or more subjects, predict the responsiveness of one or more subjects to various treatments, identify the treatment predicted to be effective for an individual subject, assign one or more subjects into an appropriate arm within a clinical trial, or a combination thereof.

The GA growth output 170 may be used to generate an output that includes an indication of whether a subject is eligible for a clinical trial for testing a medical treatment. In some embodiments, this output may be used to enroll the subject in the clinical trial, exclude the subject from participating in the clinical trial, customize a protocol in the clinical trial for the subject, or enroll the subject in a different clinical trial.

II.B.2. Example Methods with Prediction System in Training Mode

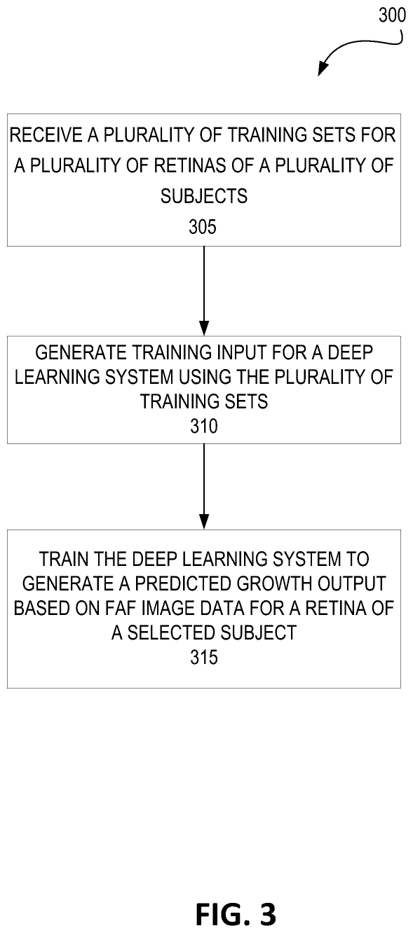

FIG. 3 is a flowchart illustrating an embodiment of a process for training a deep learning system in accordance with one or more embodiments. In one or more embodiments, method 300 may be implemented to develop a trained growth prediction system (e.g., trained growth prediction system 150 of the prediction system 100 described in FIG. 1). The process 300 in FIG. 3 is described with continuing reference to prediction system 100 of FIG. 1.

Step 305 includes receiving a plurality of training sets for a plurality of retinas of a plurality of subjects at step 305. In some embodiments and at the step 305, the plurality of training sets for a plurality of retinas of a plurality of subjects is the training dataset 163 of FIG. 1. The training dataset 163 may be, for example, accessed from a database, cloud storage, or some other type of storage. The training dataset 163 may include FAF images from multiple sources, such as, for example, clinical trials or studies, that have the same (or substantially same or similar) inclusion criteria. Ensuring that the training dataset 163 is built from studies that share the same (or the substantially the same or similar) inclusion criteria improves or increases consistency across the FAF images, which may improve training accuracy, and thereby, prediction accuracy as compared to using training sets from studies with different kinds of inclusion criteria. Prediction accuracy may be improved given that the growth image to be predicted is drawn from the same type of distribution as the training dataset (e.g., inclusion criteria for a clinical trial). In some embodiments, the growth prediction system 150 may be selected or configured such that the total amount of time, processing resources, or both used for training is reduced.

Generally, each training set of the training dataset 163 includes training FAF images for at least two different points in time. In one or more embodiments, a training set of the plurality training sets includes multiple training FAF images corresponding to different points in time (e.g., 2, 3, 4, 5, 6, or more points in time). In one or more embodiments, the training FAF images include four FAF images spaced over time by a consistent time interval (e.g., interval of 6 months) relative to a baseline point in time (e.g., time of an initial diagnosis or confirmation diagnosis of GA).

Step 310 includes generating training input for a deep learning system using the plurality of training sets. In some embodiments and at the step 310, generating training input for a deep learning system using the plurality of training sets comprises performing one or more preprocessing operations to preprocess the training FAF images in the plurality of training sets. Such preprocessing operations may include, but are not limited to, scaling, resizing, cropping, horizontal flipping, vertical flipping, adding and/or removing noise, translating, and other such preprocessing operations. In one or more embodiments, the preprocessing operations additionally include determining portions of the plurality of datasets to be used to generate the training input. This may include deleting certain FAF images from the plurality of training sets. Additional detail regarding example FAF image selection is provided in the examples below. For example, without limitation, about 80% of the plurality of datasets may be used to form the training input, while about 20% of the plurality of datasets would not be used. Generally, generating training input for a deep learning model with a plurality of datasets from multiple sources improves the predictive performance of the deep learning system. For example, using a trained deep learning model to analyze FAF images and automatically predict growth of a GA lesion may improve the speed and efficiency of making these predictions, as well as the accuracy of the predictions.

Step 315 includes generating, via a deep learning system (e.g., a deep learning model in growth prediction system 150 in FIG. 1), a predicted growth output based on FAF image data for a retina of a selected subject at step 315. In some embodiments and at the step 315, a deep learning system can be trained in a variety of different ways to create the growth prediction system 150. Additional detail regarding the different types of training is provided below.

### III. Example Experiment Using Multiple Types of Example Deep Learning Models to implement Growth Prediction System

An example experiment is associated with multiple different deep learning models for use in predicting the future region of growth of GA lesions using FAF images. These models were trained to predict GA lesion growth for various points of time. Each of these different deep learning models is one example of an implementation for growth prediction system 150 in FIG. 1.

### III.A. Example Curation of Training Dataset Via Filtering and Preprocessing to Form Training, Validation and Test Sets

The example deep learning models were trained using training input (or training dataset) (which is one example of an implementation of training dataset 163 in FIG. 1). The training dataset included imaging data derived from FAF images obtained from clinical trials (e.g., lampalizumab phase 3 clinical trials (NCT02247479 and NCT02247531) and observational studies (NCT02479386 and NCT02399072)). The study eye inclusion criteria included having well-demarcated area(s) of GA secondary to AMD with no evidence of prior or active choroidal neovascularization and having a total lesion area of 2.54 to 17.78 mm² (1-7 disc areas) residing completely within the blue-light FAF imaging field (field 2-30 degrees and image centered on the fovea), with perilesional banded or diffuse hyper-autofluorescence patterns on FAF images. If the GA lesion was multifocal, the inclusion criteria was selected such that the focal lesion was greater than 1.27 mm² (greater than 0.5 disc area). The clinical trials adhered to the Declaration of Helsinki and were Health Insurance Portability and Accountability Act compliant.

In the example experiment, macula-centered 30-degree (field 2) FAF images having 786 pixels by 786 pixels or 1536 pixels by 1536 pixels were used. Because no treatment effect was observed on lesion growth rates in the phase 3 trials, data from all treatment arms were pooled for this example analysis. GA lesion areas were graded on FAF images using RegionFinder software (Heidelberg Engineering, Inc.). Prior to starting the grading process for follow-up visit images, the human graders used automatic registration software to longitudinally register the follow-up visit FAF images to screening (e.g., baseline) visit FAF images. GA lesion areas were graded at a central reading center by at least one trained human graders (G1).

In this example experiment, the available training data includes FAF images for eyes of patients taken at various visits between screening and up to 2 years. This available training data was filtered out based on various rules to arrive at a training dataset (e.g., comprised of multiple training sets for the different patients) that could be split into training, validation, and test sets. For example, of the available training data, images of patients with missing annotations of GA lesions were excluded. Of the remaining available training data, patients with missing annotations for GA lesions at 4 consecutive longitudinal visits were excluded.

Of the remaining available training data, patients had various combinations of FAF images based on 4 consecutive visits (T1, T2, T3, T4). For example, patients had a 4-image combination for screening (e.g., the initial/baseline visit) (encoded to T1), week 24 (encoded to T2), week 48 (encoded to T3), and week 72 (encoded to T4); patients had a 4-image combination for week 24 (encoded to T1), week 48 (encoded to T2), week 72 (encoded to T3), and week 96 (encoded to T4); patients had a 4-image combination for week 48 (encoded to T1), week 72 (encoded to T2), week 96 (encoded to T3), and week 120 (encoded to T4). Thus, T1 was used to refer to the baseline visit, T2 to the 6-month visit after baseline, T3 to the 1 year visit after baseline, and T4 to the 1.5 year visit after baseline.

Of this remaining available training data, patients with misregistration of FAF images and corresponding annotations of GA lesions across longitudinal visits were excluded. Misregistration was determined based on pairwise comparisons performed between annotations at (1) T1 and T2, (2) T2 and T3, (3) T3 and T4, and (4) T2 and T4. For each of these combinations, Dice score coefficient (DSC), the change in lesion area (%), and the lesion growth rate (mm²/year) were obtained. Criteria for non-exclusion was selected such that disc (DSC)>0.7, −10%<change in lesion area (%)<100%, with all annotations being within the FAF image field of view. Cases that achieved 0.7<DSC<0.9 were manually reviewed for registration errors and peripapillary lesions, and if observed, the patient was excluded from this experiment.

Of the remaining available training data, patients that overlapped across different combinations of longitudinal visits were excluded to ensure that the final training dataset included unique patient combinations across the various combinations of longitudinal visits. In other words, no patient was duplicated across datasets.

Preprocessing included resizing all FAF images to 768 by 768 pixels. Further, each FAF image was z-normalized (a process used to normalize every pixel in an image so the mean of all values is 0 and the standard deviation is 1).

Of the final training dataset, the patient sets were split into training (310), validation (78), and tests sets (209). The training and validation sets formed therefore a developmental set. The datasets were stratified across training, validation, and test set (to ensure similar distributions in each) with respect to baseline (or initial) lesion area (e.g., large or small), lesion growth rate (e.g., fast or slow), foveal involvement (e.g., nonsubfoveal or subfoveal), focality (e.g., unifocal or multifocal), eye (e.g., right eye or left eye), study (e.g., the particular clinical study), and the visits (e.g., the particular longitudinal combination of visits).

### III.B. Example Model Development

The multiple deep learning models were each implemented using a 2-dimensional UNet (one example of a convolutional neural network (CNN)) architecture. The UNet architecture includes, for example, an encoder (contracting path), which can convert a medical image into feature maps, and a decoder (expanding path), which can convert the feature maps into a probability map of equal size to the input image. The encoder extracts image features of different spatial resolutions, which are in turn used by the decoder to derive an accurate segmentation mask.

For this example experiment, the same encoder and decoder specifications were used for all implementations of the deep learning model using a UNet. The encoder used a 34-layer residual neural network (ResNet) backbone to extract features at different resolutions, an encoder depth of 4, and pretrained ImageNet weights, where the encoder depth signifies the number of stages and the feature size decreases with each additional stage. The decoder used batch normalization between the convolutional and activation layers and had a depth of 4 with decoder channels of (128, 64, 32, 16). Training parameters included, for example, data augmentation, scheduled learning rate reductions on loss plateaus, batch size 4, maximum epochs 200 [with early stopping], and optimizer AdamW. Data augmentation consisted of randomized horizontal and vertical flipping, addition of noise, and minor translation and scaling.

Model development included the development of three different whole-lesion models as well as three multiclass models (e.g., all developed using PyTorch). Hyperparameter optimization was conducted across learning rate (1e−3, 1e−4, 5e−4, 1e−5), weight decay (1e−2, 1e−4), and loss functions via a grid search, with 48 separate models being developed for each model type (6 total types). The search was designed to increase or maximize the region of growth disc, which was calculated during postprocessing. The performance of the models was evaluated by calculating the Dice score, coefficient of determination (R²), and the squared Pearson correlation coefficient (r²) between the true and derived GA lesion 1 year region of growth.

Image segmentation may be performed to improve the accuracy of the deep learning model(s) 150. Segmentation may be performed using, for example, an autoregressive network (which can be used to predict semantic segmentation maps of unobserved future frames from past sequences of videos), a temporal encoder-decoder network architecture (which can be used to derive features from past frames and later construct the future semantic segmentation), a separate encoder-decoder architecture (which may be used to identify future trajectory points of objects), an LSTM component (e.g., the LSTM component in any of the aforementioned architectures), or a combination thereof may be used to predict future lesion growth and/or potentially concurrent lesion growth across patient visits. As one example, an autoregressive segmentation model underpinned on LSTMs may be used to predict future, and potentially concurrent, lesion growth across patient visits.

### III.B.1. Example Whole-Lesion Models

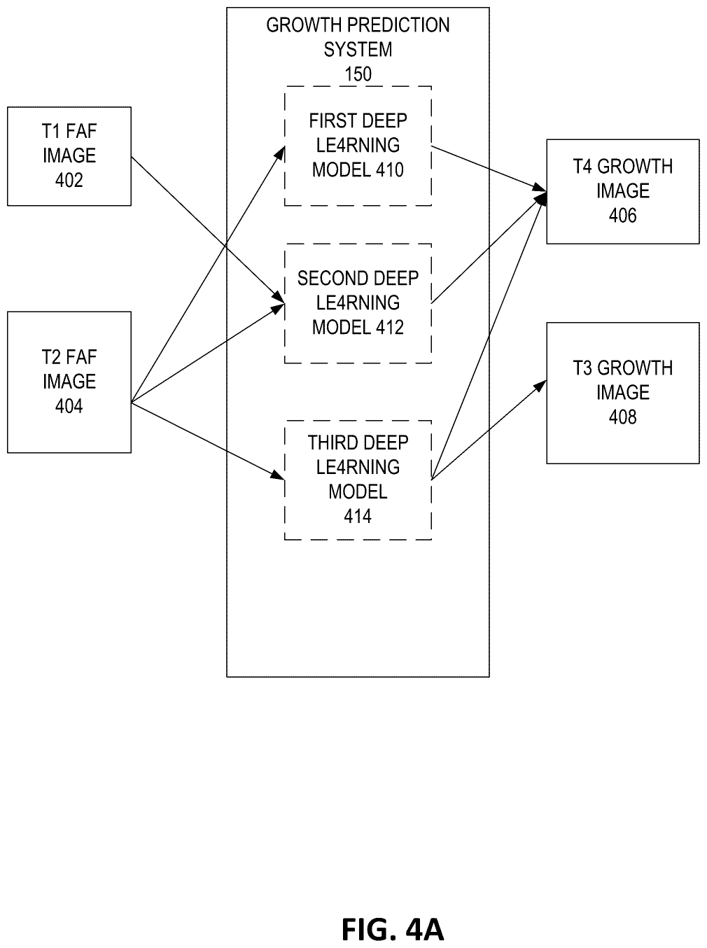

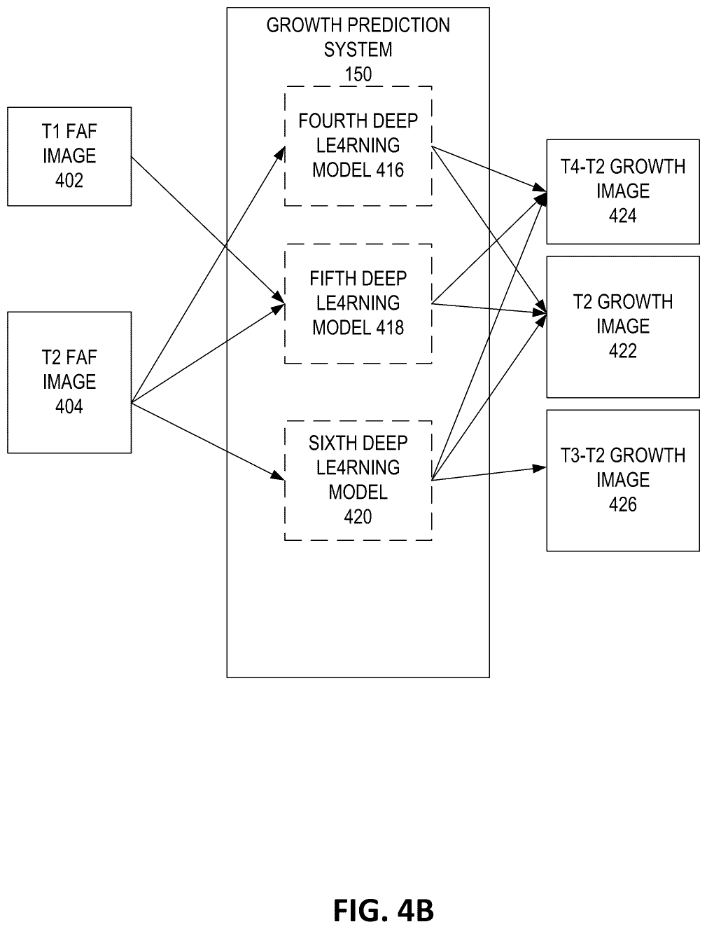

The three example whole-lesion models are described with respect to FIG. 4.

FIG. 4A is a schematic diagram of different example deep learning models used to implement the growth prediction system 150 from FIG. 1 in accordance with one or more embodiments. In FIG. 4A, growth prediction system 150 is implemented in different ways and trained to receive FAF images that capture GA lesions at various points in time and generate predicted growth outputs (e.g., including growth images) for various future points in time. These various points in time may include, for example, T1, T2, T3, and T4 as described above, with these time points being spaced apart by a consistent time interval of 6 months.

In this example experiment, T1 FAF image 402 is an example of one type of image input generated at a first point in time (T1) that may be sent into growth prediction system 150. T1 FAF image 402 may be one example of an implementation for modified FAF image 145 in FIG. 1. T1 for T1 FAF image 402 is a baseline point in time. T2 FAF image is an example of one type of image input generated at a second point in time (T2) that may be sent into growth prediction system 150. T2 FAF 404 image may be one example of an implementation for modified FAF image 145 in FIG. 1. T2 for T2 FAF image 402 is 6 months after baseline.

In this example experiment, growth prediction system 150 is implemented using first deep learning model 410 (i.e., model 1). First deep learning model 410 is implemented using a Simple UNet architecture trained to infer the T4 whole GA lesion from the T2 FAF image 404. More specifically, first deep learning model 410 receives and processes T2 FAF image 404 to generate T4 growth image 406. T4 growth image 406 may be one example of an implementation for growth image 175 in FIG. 1. T4 growth image 406 is a growth image illustrating the total area of the retina predicted to be affected by the GA lesion at T4.

In this example experiment, growth prediction system 150 is also implemented using second deep learning model 414 (i.e., model 2). Second deep learning model 414 is implemented using a Multichannel UNet, trained to infer the T4 whole GA lesion from the combination of the T1 FAF image 402 and T2 FAF image 404.

In this example experiment, growth prediction system 150 is also implemented using third deep learning model 414 (i.e., model 3). Third deep learning model 414 is implemented using a Sequential Label UNet, trained to infer T3 growth image 408 and T4 growth image 406 from the T2 FAF image 404. T3 growth image 408 may be one example of an implementation for growth image 175 in FIG. 1. T3 growth image 408 is a growth image illustrating the total area of the retina predicted to be affected by the GA lesion at T3.

These three whole lesion models were trained using loss functions that included the Dice, Dice cross-entropy, Tversky (a=0.6, b=0.4), Tversky (a=0.4, b=0.6), and focal losses.

### III.B.2. Example Multi-Class Models

The three example whole-lesion models are described with respect to FIG. 4B.

FIG. 4B is a schematic diagram of different example deep learning models used to implement the growth prediction system 150 from FIG. 1 in accordance with one or more embodiments. In FIG. 4B, growth prediction system 150 is implemented in different ways and trained to receive FAF images that capture GA lesions at various points in time and generate predicted growth outputs (e.g., including growth images) for various future points in time. These various points in time may include, for example, T1, T2, T3, and T4 as described above, with these time points being spaced apart by a consistent time interval of 6 months.

In this example experiment, growth prediction system 150 is also implemented using fourth deep learning model 416 (i.e., model 4), fifth deep learning model 418 (i.e. model 5), and sixth deep learning model 420 (i.e., model 6.

Fourth deep learning model 416 is implemented using a Simple UNet, trained to infer T2 growth image 422 and T4−T2 growth image 424 using T2 FAF image 404. T2 growth image 422 illustrates the predicted whole lesion at T2. T4−T2 growth image 424 illustrates the region representing new growth between T4 and T2. In other words, T4−T2 growth image 424 may indicate the difference between the predicted GA lesion at time T4 and time T2.

Fifth deep learning model 418 is implemented using a Multichannel UNet, trained to infer T2 growth image 422 and T4−T2 growth image 424 using T2 FAF image 404 and T1 FAF image. 402.

Sixth deep learning model 420 is implemented using a Sequential UNet, trained to infer T2 growth image 422, T4−T2 growth image 424, and T3−T2 growth image 426 using T2 FAF image 404. T3−T2 growth image 426 illustrates the region representing new growth between T3 and T2. In other words, T3−T2 growth image 426 may indicate the difference between the predicted GA lesion at time T3 and time T2.

These three multiclass models were trained using loss functions that included the Dice, generalized Dice, generalized Dice focal, Dice cross-entropy, Dice focal, and focal losses.

### III.B.3. Example Postprocessing for Whole Lesion and Multiclass Models

Post-processing of the three example whole lesion models included applying the sigmoid function to each prediction and setting the threshold at >0.5 to define the final resultant prediction. The 1-year ROG of GA was derived by taking the difference between the predicted T4 lesion and grader T2 lesion annotation. This post-processing method was just one example of how post-processing may have been performed

Post-processing of the three example whole lesion models included, for inference, applying the softmax function channel-wise to each prediction with the highest probability defining the final prediction. The background (e.g., image background that was not considered GA lesion) was considered a separate class, as during training. For fourth deep learning model 416 and fifth deep learning model 418, the T4−T2 growth image 424 was predicted and no further postprocessing was required. For sixth deep learning model 420, the T4−T2 growth image 424 was generated by adding the T3−T2 growth image 426 and a separate, T4−T3 growth image.

Post-processing for the multiclass models also enabled generation of predicted whole lesion growth images (e.g., precited total lesion area at future point in time). For example, to obtain the total predicted GA lesion at T4, the T2 growth image 422 was summed with the T4−T2 growth image 424. Thus, these three multiclass models also allowed the generation of predicted whole lesion at T4 and may be referred to as multiclass whole lesion models when used in this manner. This method was implemented to reduce the variability in the region of growth prediction coming from the predicted T2 lesion.

### III.C. Example Results

Analyzing the results included analyzing squared Pearson correlation coefficient (r²) and estimating a linear calibration function (using population least squares linear regression) from the validation set. The predictions in the test set were transformed with the calibration function to obtain recalibrated predictions.

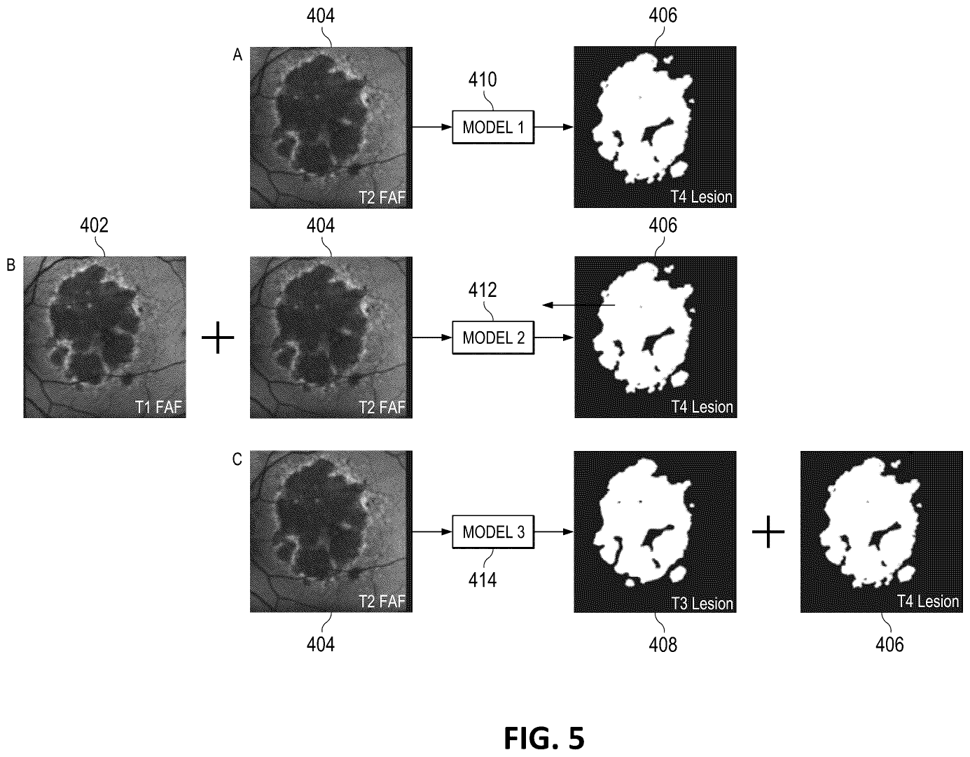

FIG. 5 is an example workflow showing example processing for the three whole lesion models in accordance with one or more embodiments. For example, FIG. 5 illustrates examples of the input FAF images that may be sent into the whole lesion models (e.g., first deep learning model 410, second deep learning model 412, third deep learning model 414) and the outputs that may be generated as described with respect to FIG. 4A above.

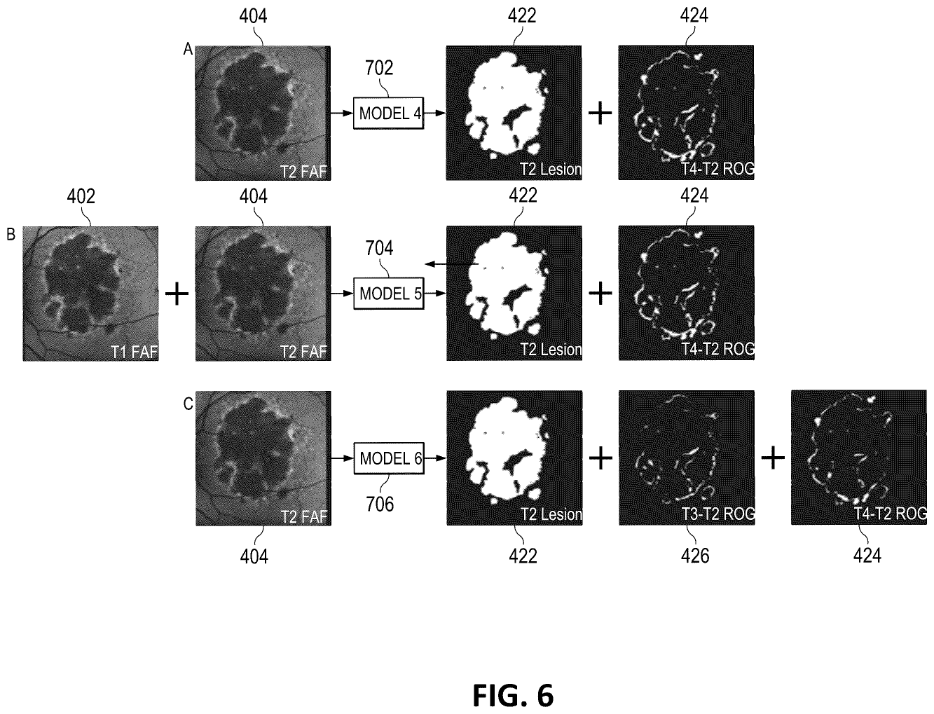

FIG. 6 is an example workflow showing example processing for the three multiclass models in accordance with one or more embodiments. For example, FIG. 6 illustrates examples of the input FAF images that may be sent into the whole lesion models (e.g., fourth deep learning model 416, fifth deep learning model 418, sixth deep learning model 420) and the outputs that may be generated as described with respect to FIG. 4B above.

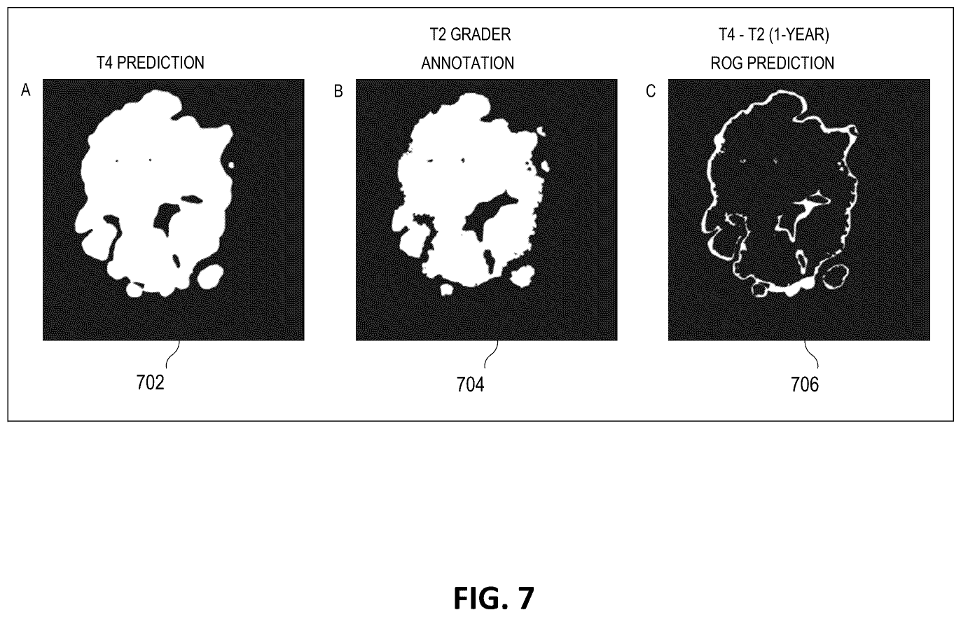

FIG. 7 illustrates example images for an example workflow for post-processing for whole lesion models in accordance with one or more example embodiments. In FIG. 7, image 702 is the T4 whole lesion prediction generated by a whole lesion model, image 704 is the T2 annotation of whole lesion by a human grader, and image 706 is the final predicted region of growth between T4 and T2 (T4−T2) generated by subtracting image 704 from image 702.

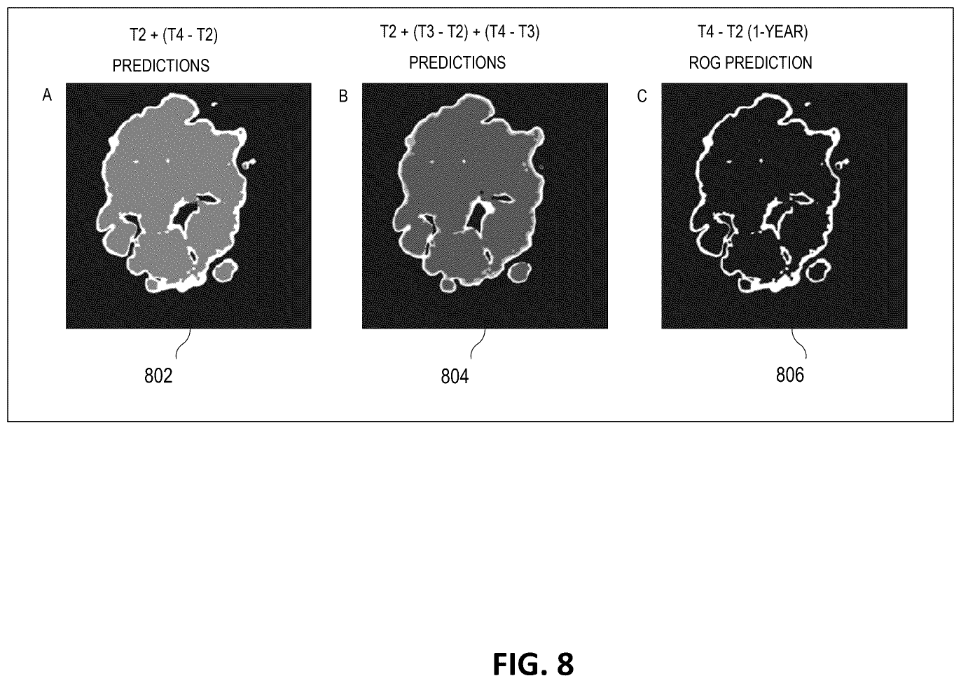

FIG. 8 illustrates example images for an example workflow for post-processing for multiclass models in accordance with one or more example embodiments. In FIG. 8, image 802 is an example of the T2 whole lesion and T4−T2 ROG predictions that can be produced by fourth deep learning model 416 and fifth deep learning model 418. Image 804 is an example of the T2 whole lesion, T3−T2 ROG and T4−T3 ROG predictions that can be produced by sixth deep learning model 420. Image 806 is the T4−T2 prediction (ROG) that can be taken directly from the T4−T2 prediction or by addition for T3−T2 and T4−T3 predictions.

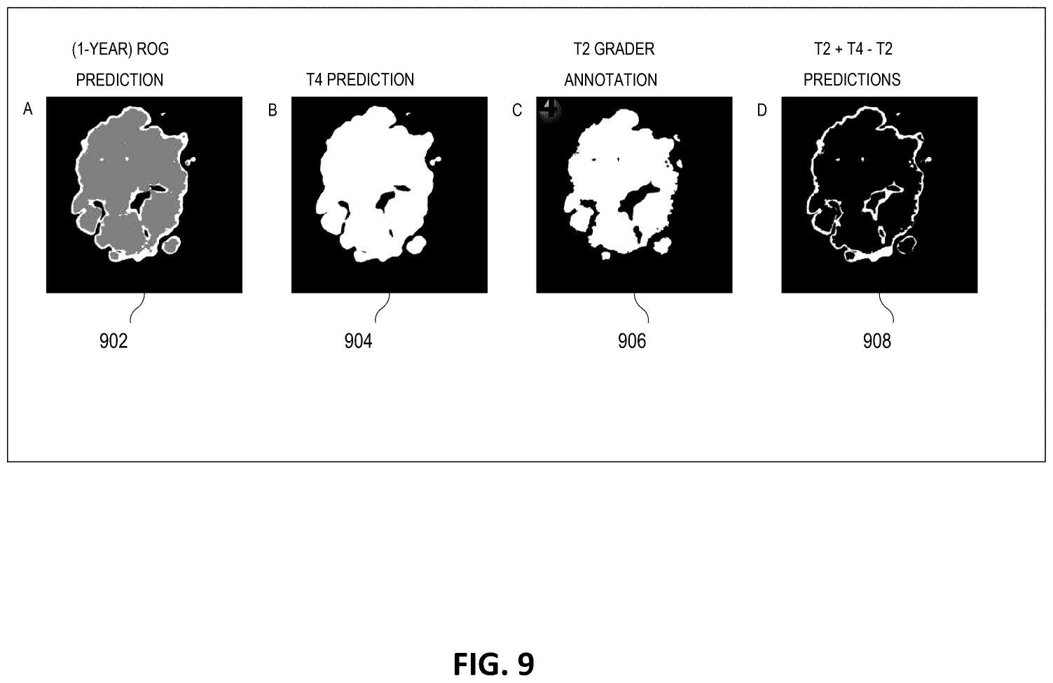

FIG. 9 illustrates example images for an example workflow for post-processing of multiclass whole lesion models in accordance with one or more example embodiments. In FIG. 9, image 902 is the T2 whole lesion and T4−T2 predictions that can be produced by fourth deep learning model 416 and fifth deep learning model 418. Image 904 is the summation of T2 whole lesion and T4−T2 ROG predictions to generate T4 whole lesion prediction. Image 906 is the T2 annotation by a human grader (G1). Image 908 is the T4−T2 prediction (ROG) generated by subtracting the T2 annotation from the T4 prediction.

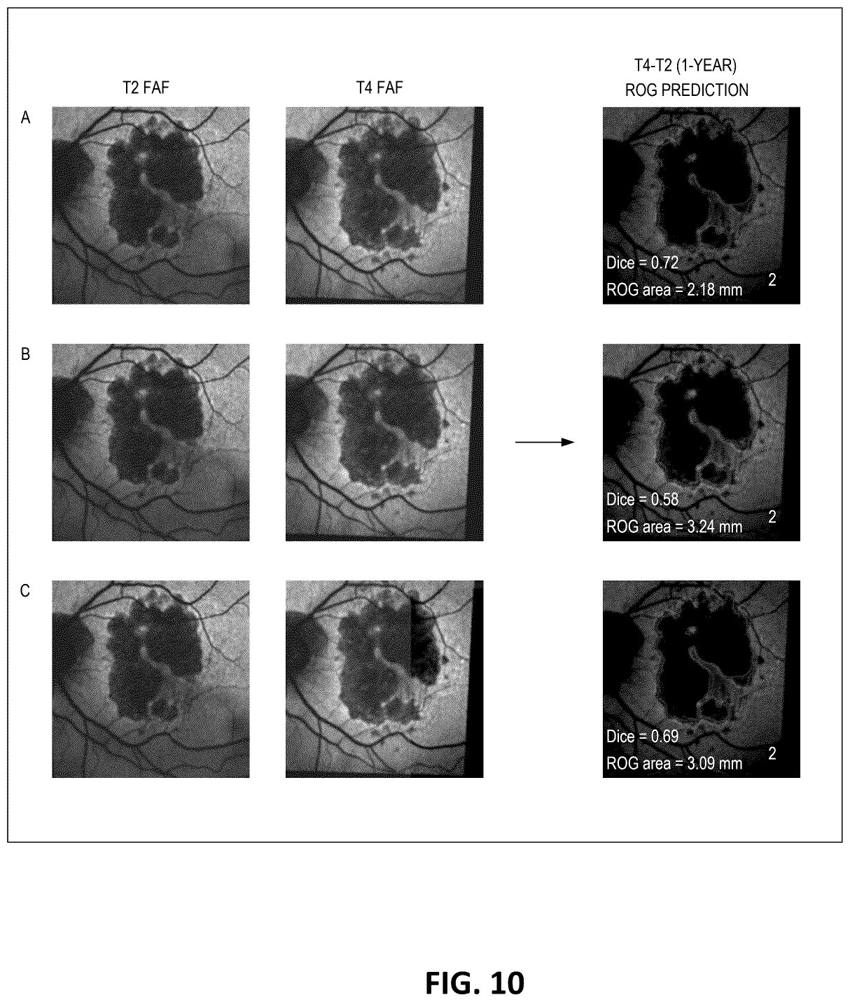

FIG. 10 illustrates examples of the predicted growth images generated for different deep learning models in accordance with one or more embodiments. The A set of images show the predicted 1-year ROG (e.g., T4−T2 growth image) predicted with respect to an example input T2 FAF image and shows the ground truth T4 FAF image for second deep learning model 412. The B set of images show the predicted 1-year ROG (e.g., T4−T2 growth image) predicted with respect to an example input T2 FAF image and shows the ground truth T4 FAF image for fifth deep learning model 418 implemented just for multiclass. The C set of images show the predicted 1-year ROG (e.g., T4−T2 growth image) predicted with respect to an example input T2 FAF image and shows the ground truth T4 FAF image for fifth deep learning model 418 implemented as multiclass whole lesion.

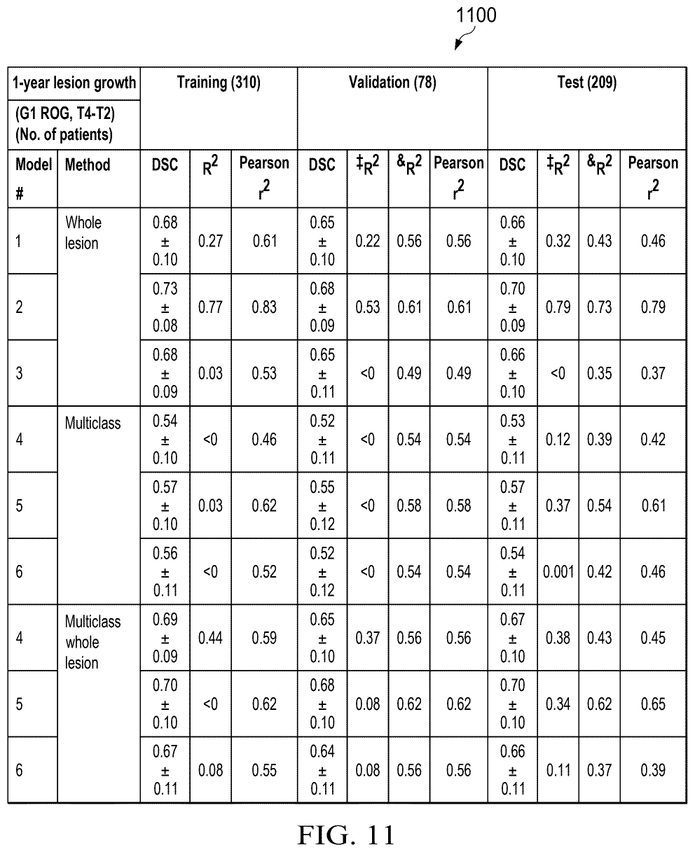

FIG. 11 is a table illustrating various results of the example experiment for each of the different six models for the training, validation, and test sets in accordance with one or more embodiments.

### IV. Example Output Use Cases

Generally, the prediction system 100 and/or the growth prediction system 150 that predicts GA growth output at a future point in time, wherein the GA growth output includes a visual illustration (e.g., graphical indication) of the predicted location(s) relative to a retina of a subject, is an improvement to the technical field of GA lesion prediction and treatment because of the reduction in cost, time, expense, and/or computing resources that may be achieved. Further, in some cases, the predicted GA growth output may be generated at least as if not more accurately and/or reliably than human graders such that the growth prediction system 150 may be successfully relied upon for use in clinical practice. For example, the GA growth output 170, including the mask 185, may be used to determine whether a subject is a candidate for a clinical trial, to which clinical trial to assign the subject, how to customize a treatment for the subject, how to monitor the progress of the subject during the clinical trial, or a combination thereof. The techniques described herein can be used to predict the prognosis of one or more subjects, predict the responsiveness of one or more subjects to various treatments, identify the treatment predicted to be effective for an individual subject, assign one or more subjects into an appropriate arm within a clinical trial, or a combination thereof.

Generally, the prediction system 100 and/or the growth prediction system 150 that predicts GA growth output at a future point in time, wherein the GA growth output includes a visual illustration of the predicted location(s) relative to a retina of a subject, is an improvement to the technical field of GA lesion prediction and treatment because the predicted GA growth output may be used to generate an output that includes an indication of whether a subject is eligible for a clinical trial for testing a medical treatment for geographic atrophy. In some embodiments, this output may be used to enroll the subject in the clinical trial, exclude the subject from participating in the clinical trial, customize a protocol in the clinical trial for the subject, or enroll the subject in a different clinical trial.

Generally, the prediction system 100 and/or the growth prediction system 150 that predicts GA growth output at a future point in time, wherein the GA growth output includes a visual illustration of the predicted location(s) relative to a retina of a subject, is an improvement to the technical field of GA lesion prediction and treatment because the predicted GA growth output is generated with an accuracy that can be successfully relied upon for use in clinical practice. For example, the GA growth output 170, including the mask 185, may be used to determine whether a subject is a candidate for a clinical trial, to which clinical trial to assign the subject, how to customize a treatment for the subject, how to monitor the progress of the subject during the clinical trial, or a combination thereof. The techniques described herein can be used to predict the prognosis of one or more subjects, predict the responsiveness of one or more subjects to various treatments, identify the treatment predicted to be effective for an individual subject, assign one or more subjects into an appropriate arm within a clinical trial, or a combination thereof.

Generally, the prediction system 100 and/or the growth prediction system 150 that predicts GA growth output at a future point in time provides a technical effect of identifying and generating a GA growth output that includes a visual illustration of the predicted location(s) relative to a retina of a subject.

### V. Example of Computer-Implemented System

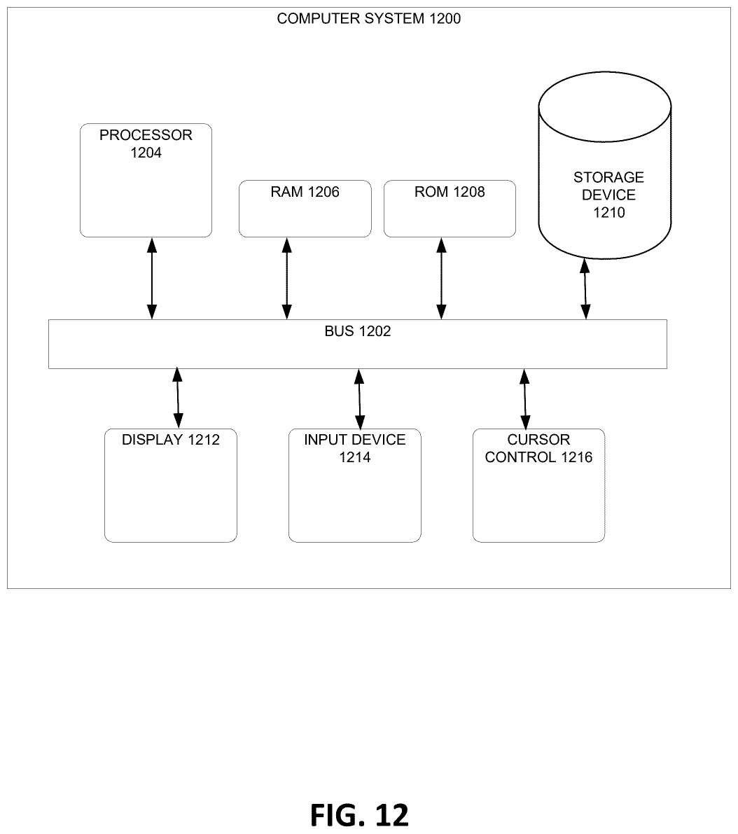

FIG. 12 is a block diagram of a computer system in accordance with various embodiments. Computer system 1200 may be an example of one implementation for computing platform 105 described above in FIG. 1.

In one or more examples, computer system 1200 can include a bus 1202 or other communication mechanism for communicating information, and a processor 1204 coupled with the bus 1202 for processing information. In various embodiments, the computer system 1200 can also include a memory, which can be a random-access memory (RAM) 1206 or other dynamic storage device, coupled to the bus 1202 for determining instructions to be executed by the processor 1204. Memory also can be used for storing temporary variables or other intermediate information during execution of instructions to be executed by the processor 1204. In various embodiments, the computer system 1200 can further include a read only memory (ROM) 1208 or other static storage device coupled to the bus 1202 for storing static information and instructions for the processor 1204. A storage device 1210, such as a magnetic disk or optical disk, can be provided and coupled to the bus 1202 for storing information and instructions.

In various embodiments, the computer system 1200 can be coupled via the bus 1202 to a display 1212, such as a cathode ray tube (CRT) or liquid crystal display (LCD), for displaying information to a computer user. An input device 1214, including alphanumeric and other keys, can be coupled to the bus 1202 for communicating information and command selections to the processor 1204. Another type of user input device is a cursor control 1216, such as a mouse, a joystick, a trackball, a gesture input device, a gaze-based input device, or cursor direction keys for communicating direction information and command selections to the processor 1204 and for controlling cursor movement on the display 1212. This input device 1216 typically has two degrees of freedom in two axes, a first axis (e.g., x) and a second axis (e.g., y), that allows the device to specify positions in a plane. However, it should be understood that input devices 1214 allowing for three-dimensional (e.g., x, y, and z) cursor movement are also contemplated herein.

Consistent with certain implementations of the present teachings, results can be provided by the computer system 1200 in response to the processor 1204 executing one or more sequences of one or more instructions contained in the RAM 1206. Such instructions can be read into the RAM 1206 from another computer-readable medium or computer-readable storage medium, such as the storage device 1210. Execution of the sequences of instructions contained in the RAM 1206 can cause the processor 1204 to perform the processes described herein. Alternatively, hard-wired circuitry can be used in place of or in combination with software instructions to implement the present teachings. Thus, implementations of the present teachings are not limited to any specific combination of hardware circuitry and software.

The term “computer-readable medium” (e.g., data store, data storage, storage device, data storage device, etc.) or “computer-readable storage medium” as used herein refers to any media that participates in providing instructions to the processor 1204 for execution. Such a medium can take many forms, including but not limited to, non-volatile media, volatile media, and transmission media. Examples of non-volatile media can include, but are not limited to, optical, solid state, magnetic disks, such as the storage device 1210. Examples of volatile media can include, but are not limited to, dynamic memory, such as the RAM 1206. Examples of transmission media can include, but are not limited to, coaxial cables, copper wire, and fiber optics, including the wires that comprise the bus 1202.

Common forms of computer-readable media include, for example, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, any other memory chip or cartridge, or any other tangible medium from which a computer can read.

In addition to computer readable medium, instructions or data can be provided as signals on transmission media included in a communications apparatus or system to provide sequences of one or more instructions to the processor 1204 of the computer system 1200 for execution. For example, a communication apparatus may include a transceiver having signals indicative of instructions and data. The instructions and data are configured to cause one or more processors to implement the functions outlined in the disclosure herein. Representative examples of data communications transmission connections can include, but are not limited to, telephone modem connections, wide area networks (WAN), local area networks (LAN), infrared data connections, NFC connections, optical communications connections, etc.

It should be appreciated that the methodologies described herein, flowcharts, diagrams, and accompanying disclosure can be implemented using the computer system 1200 as a standalone device or on a distributed network of shared computer processing resources such as a cloud computing network.

The methodologies described herein may be implemented by various means depending upon the application. For example, these methodologies may be implemented in hardware, firmware, software, or any combination thereof. For a hardware implementation, the processing unit may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, electronic devices, other electronic units designed to perform the functions described herein, or a combination thereof.

In various embodiments, the methods of the present teachings may be implemented as firmware and/or a software program and applications written in conventional programming languages such as C, C++, Python, etc. If implemented as firmware and/or software, the embodiments described herein can be implemented on a non-transitory computer-readable medium in which a program is stored for causing a computer to perform the methods described above. It should be understood that the various engines described herein can be provided on a computer system, such as the computer system 1200, whereby the processor 1204 would execute the analyses and determinations provided by these engines, subject to instructions provided by any one of, or a combination of, the memory components the RAM 1206, the ROM, 1208, or the storage device 1210 and user input provided via the input device 1214.

### VI. Recitation of Embodiments

Embodiment 1: A method comprising: receiving fundus autofluorescence (FAF) image data for a retina of a subject; wherein the FAF image data includes a first FAF image associated with a first point in time; generating an image input for a deep learning system using the FAF image data; and generating, via the deep learning system, a predicted growth output for a geographic atrophy (GA) lesion in the retina using the image input; wherein the predicted growth output is associated with at least one future point in time after the first point in time.

Embodiment 2: The method of Embodiment 1, wherein the predicted growth output comprises a first growth image that illustrates a first predicted region of growth for the GA lesion with respect to the retina of the subject between a first reference point in time and a first future point in time after the first reference point in time.

Embodiment 3: The method of Embodiment 2, wherein the predicted growth output further comprises a second growth image that illustrates a second predicted region of growth for the GA lesion with respect to the retina of the subject between a second reference point in time and a second future point in time after the second reference point in time

Embodiment 4: The method of Embodiment 3, wherein: the second reference point in time and the first reference point in time are a same point in time or different points in time; and the second future point in time is different from the first future point in time.

Embodiment 5: The method of any one of Embodiments 2-4, wherein the first reference point in time is the first point in time or a point in time between the first point in time and the first future point in time.

Embodiment 6: The method of any one of Embodiments 1-5, wherein the predicted growth output comprises a growth image that illustrates an area of the retina predicted to be affected by the GA lesion at a selected future point in time.

Embodiment 7: The method of any one of Embodiments 1-6, wherein the predicted growth output comprises a computed area for an entire area of the retina in an FAF image of the FAF image data predicted to be affected by the GA lesion at a selected future point in time.

Embodiment 8: The method of any one of Embodiments 1-7, wherein the predicted growth output comprises a computed area for an entire area of the retina in an FAF image of the FAF image data predicted to be affected by the GA lesion at a selected future point in time.

Embodiment 9: The method of Embodiment 1, wherein the predicted growth output comprises a growth image that illustrates an area for new growth of the GA lesion between two points in time.

Embodiment 10: The method of Embodiment 9, wherein the predicted growth output comprises a computed area for new growth between two points in time.

Embodiment 11: The method of any one of Embodiments 1-10, wherein the FAF image data further includes a second FAF image associated with a second point in time that is after the first point in time; and wherein generating the image input comprises: preprocessing each of the first FAF image and the second FAF image such that the image input includes a first preprocessed FAF image and a second preprocessed FAF image.

Embodiment 12: The method of Embodiment 10, wherein the predicted growth output comprises a growth image illustrating a predicted region of growth for the GA lesion with respect to the retina of the subject with respect to a selected future point in time; and wherein the growth image comprises: an image background associated with the first FAF image or the second FAF image; and a mask over the image background, wherein the mask identifies the predicted region of growth relative to the retina of the subject with respect to the selected future point in time.

Embodiment 13: The method of Embodiment 10, wherein the deep learning system comprises a trained long-short term memory convolutional neural network.

Embodiment 14: The method of any one of Embodiments 1-13, wherein the deep learning system comprises a trained convolutional neural network (CNN); wherein a training dataset used to train the trained CNN comprises a plurality of training sets corresponding respectively to a plurality of eyes; and wherein a training set of the plurality training sets comprises training FAF images corresponding to four different points in time.

Embodiment 15: The method of Embodiment 14, wherein the training FAF images of the training dataset are stratified by at least one of baseline lesion area, lesion growth rate, foveal involvement, or focality.

Embodiment 16: The method of Embodiment 14 or 15, wherein the training FAF images of the training set comprise four FAF images spaced over time by a consistent time interval.

Embodiment 17: A method of training a deep learning system comprising: generating training input for a deep learning system using the plurality of training sets; and training the deep learning system to generate a predicted growth output based on FAF image data for a retina of a selected subject, wherein the predicted growth output indicates a predicted growth of a geographic atrophy (GA) lesion in the retina with respect to at least one future point in time.

Embodiment 18: The method of Embodiment 17, wherein the predicted growth output comprises a growth image illustrating an area of the retina predicted to be affected by the GA lesion with respect to a future point in time.

Embodiment 19: The method of Embodiment 17 or 18, wherein the predicted growth output comprises a computed area for an entire area of the retina predicted to be affected by the GA lesion with respect to a future point in time.

Embodiment 20: A system comprising: one or more data processors; and a non-transitory computer readable storage medium containing instructions which, when executed on the one or more data processors, cause the one or more data processors to perform the method of any one of Embodiments 1-19.

Embodiment 21: A computer-program product tangibly embodied in a non-transitory machine-readable storage medium, including instructions configured to cause one or more data processors to perform the method of any one of Embodiments 1-19.

The disclosure is not limited to these example embodiments described herein and various configurations and implementations of the elements, components, models, and steps described herein may be used to perform GA growth prediction.

### VII. Exemplary Context and Definitions

The disclosure is not limited to these example embodiments and applications or to the manner in which the example embodiments and applications operate or are described herein. Moreover, the figures may show simplified or partial views, and the dimensions of elements in the figures may be exaggerated or otherwise not in proportion.

Unless otherwise defined, scientific and technical terms used in connection with the present teachings described herein shall have the meanings that are commonly understood by those of ordinary skill in the art. Further, unless otherwise required by context, singular terms shall include pluralities and plural terms shall include the singular. Generally, nomenclatures utilized in connection with, and techniques of, chemistry, biochemistry, molecular biology, pharmacology, and toxicology are described herein are those well-known and commonly used in the art.

As the terms “on,” “attached to,” “connected to,” “coupled to,” or similar words are used herein, one element (e.g., a component, a material, a layer, a substrate, etc.) can be “on,” “attached to,” “connected to,” or “coupled to” another element regardless of whether the one element is directly on, attached to, connected to, or coupled to the other element or there are one or more intervening elements between the one element and the other element. In addition, where reference is made to a list of elements (e.g., elements a, b, c), such reference is intended to include any one of the listed elements by itself, any combination of less than all of the listed elements, and/or a combination of all of the listed elements. Section divisions in the specification are for ease of review only and do not limit any combination of elements discussed.

The term “subject” may refer to a subject of a clinical trial, a person undergoing treatment, a person undergoing anti-cancer therapies, a person being monitored for remission or recovery, a person undergoing a preventative health analysis (e.g., due to their medical history), or any other person or patient of interest. In various cases, “subject” and “patient” may be used interchangeably herein.

As used herein, “substantially” means sufficient to work for the intended purpose. The term “substantially” thus allows for minor, insignificant variations from an absolute or perfect state, dimension, measurement, result, or the like such as would be expected by a person of ordinary skill in the field but that do not appreciably affect overall performance. When used with respect to numerical values or parameters or characteristics that can be expressed as numerical values, “substantially” means within ten percent.

The term “ones” means more than one.

As used herein, the term “plurality” may be 2, 3, 4, 5, 6, 7, 8, 9, 10, or more.

As used herein, the term “set of” means one or more. For example, a set of items includes one or more items.

As used herein, the phrase “at least one of,” when used with a list of items, means different combinations of one or more of the listed items may be used and only one of the items in the list may be needed. The item may be a particular object, thing, step, operation, process, or category. In other words, “at least one of” means any combination of items or number of items may be used from the list, but not all of the items in the list may be required. For example, without limitation, “at least one of item A, item B, or item C” means item A; item A and item B; item B; item A, item B, and item C; item B and item C; or item A and C. In some cases, “at least one of item A, item B, or item C” means, but is not limited to, two of item A, one of item B, and ten of item C; four of item B and seven of item C; or some other suitable combination.

As used herein, a “model” may include one or more algorithms, one or more mathematical techniques, one or more machine learning algorithms, or a combination thereof.

As used herein, “machine learning” may be the practice of using algorithms to parse data, learn from it, and then make a determination or prediction about something in the world. Machine learning uses algorithms that can learn from data without relying on rules-based programming.

As used herein, an “artificial neural network” or “neural network” (NN) may refer to mathematical algorithms or computational models that mimic an interconnected group of artificial nodes or neurons that processes information based on a connectionist approach to computation. Neural networks, which may also be referred to as neural nets, can employ one or more layers of nonlinear units to predict an output for a received input. Some neural networks include one or more hidden layers in addition to an output layer. The output of each hidden layer is used as input to the next layer in the network, i.e., the next hidden layer or the output layer. Each layer of the network generates an output from a received input in accordance with current values of a respective set of parameters. In the various embodiments, a reference to a “neural network” may be a reference to one or more neural networks.

A neural network may process information in two ways: when it is being trained it is in training mode and when it puts what it has learned into practice it is in inference (or prediction) mode. Neural networks learn through a feedback process (e.g., backpropagation) which allows the network to adjust the weight factors (modifying its behavior) of the individual nodes in the intermediate hidden layers so that the output matches the outputs of the training data. In other words, a neural network learns by being fed training data (learning examples) and eventually learns how to reach the correct output, even when it is presented with a new range or set of inputs. A neural network may include, for example, without limitation, at least one of a Feedforward Neural Network (FNN), a Recurrent Neural Network (RNN), a Modular Neural Network (MNN), a Convolutional Neural Network (CNN), a Residual Neural Network (ResNet), an Ordinary Differential Equations Neural Networks (neural-ODE), LSTM, or another type of neural network.

As used herein, a “lesion” may be a region in an organ or tissue that has suffered damage via injury or disease. This region may be a continuous or discontinuous region. For example, as used herein, a lesion may include multiple regions. A geographic atrophy (GA) lesion is a region of the retina that has suffered chronic progressive degeneration. As used herein, a GA lesion may include one lesion (e.g., one continuous lesion region) or multiple lesions (e.g., discontinuous lesion region comprised of multiple, separate lesions).

As used herein, a “lesion area” may be the total area covered by a lesion, whether that lesion be a continuous region or a discontinuous region.

As used herein, “longitudinal” may refer to over a period of time. The period of time may be in days, weeks, months, years, or some other measure of time.

As used herein, a “growth rate” corresponding to a GA lesion may be a longitudinal change in the lesion area of the GA lesion. In other words, the “growth rate” may be a change in lesion area over time. In some cases, this growth rate may be an annualized growth rate. This growth rate may also be referred to as a lesion growth rate or a GA growth rate.

### VIII. Additional Considerations

Any headers and/or subheaders between sections and subsections of this document are included solely for the purpose of improving readability and do not imply that features cannot be combined across sections and subsection. Accordingly, sections and subsections do not describe separate embodiments.

While the present teachings are described in conjunction with various embodiments, it is not intended that the present teachings be limited to such embodiments. On the contrary, the present teachings encompass various alternatives, modifications, and equivalents, as will be appreciated by those of skill in the art. The present description provides preferred example embodiments, and is not intended to limit the scope, applicability, or configuration of the disclosure. Rather, the present description of the preferred example embodiments will provide those skilled in the art with an enabling description for implementing various embodiments. It is understood that various changes may be made in the function and arrangement of elements without departing from the spirit and scope as set forth in the appended claims. Thus, such modifications and variations are considered to be within the scope set forth in the appended claims. Further, the terms and expressions which have been employed are used as terms of description and not of limitation, and there is no intention in the use of such terms and expressions of excluding any equivalents of the features shown and described or portions thereof, but it is recognized that various modifications are possible within the scope of the disclosure.

In describing the various embodiments, the specification may have presented a method and/or process as a particular sequence of steps. However, to the extent that the method or process does not rely on the particular order of steps set forth herein, the method or process should not be limited to the particular sequence of steps described, and one skilled in the art can readily appreciate that the sequences may be varied and still remain within the spirit and scope of the various embodiments.

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

Specific details are given in the present description to provide an understanding of the embodiments. However, it is understood that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

1. A method comprising:
receiving fundus autofluorescence (FAF) image data for a retina of a subject;
wherein the FAF image data includes a first FAF image associated with a first point in time;
generating an image input for a deep learning system using the FAF image data; and
generating, via the deep learning system, a predicted growth output for a geographic atrophy (GA) lesion in the retina using the image input;
wherein the predicted growth output is associated with at least one future point in time after the first point in time.

2. The method of claim 1, wherein the predicted growth output comprises a first growth image that illustrates a first predicted region of growth for the GA lesion with respect to the retina of the subject between a first reference point in time and a first future point in time after the first reference point in time.

3. The method of claim 2, wherein the predicted growth output further comprises a second growth image that illustrates a second predicted region of growth for the GA lesion with respect to the retina of the subject between a second reference point in time and a second future point in time after the second reference point in time.

4. The method of claim 3, wherein:
the second reference point in time and the first reference point in time are a same point in time or different points in time; and
the second future point in time is different from the first future point in time.

5. The method of claim 2, wherein the first reference point in time is the first point in time or a point in time between the first point in time and the first future point in time.

6. The method of claim 1, wherein the predicted growth output comprises a growth image that illustrates an area of the retina predicted to be affected by the GA lesion at a selected future point in time.

7. The method of claim 1, wherein the predicted growth output comprises a computed area for an entire area of the retina in an FAF image of the FAF image data predicted to be affected by the GA lesion at a selected future point in time.

8. The method of claim 1, wherein the predicted growth output comprises a growth image that illustrates an area for new growth of the GA lesion between two points in time.

9. The method of claim 1, wherein the predicted growth output comprises a computed area for new growth between two points in time.

10. The method of claim 1, wherein the FAF image data further includes a second FAF image associated with a second point in time that is after the first point in time; and
wherein generating the image input comprises:
preprocessing each of the first FAF image and the second FAF image such that the image input includes a first preprocessed FAF image and a second preprocessed FAF image.

11. The method of claim 10,
wherein the predicted growth output comprises a growth image illustrating a predicted region of growth for the GA lesion with respect to the retina of the subject with respect to a selected future point in time; and
wherein the growth image comprises:
an image background associated with the first FAF image or the second FAF image; and
a mask over the image background, wherein the mask identifies the predicted region of growth relative to the retina of the subject with respect to the selected future point in time.

12. The method of claim 1, wherein the deep learning system comprises a trained long-short term memory convolutional neural network.

13. The method of claim 1,
wherein the deep learning system comprises a trained convolutional neural network (CNN);
wherein a training dataset used to train the trained CNN comprises a plurality of training sets corresponding respectively to a plurality of eyes; and
wherein a training set of the plurality training sets comprises training FAF images corresponding to four different points in time.

14. The method of claim 13, wherein the training FAF images of the training dataset are stratified by at least one of baseline lesion area, lesion growth rate, foveal involvement, or focality.

15. The method of claim 14, wherein the training FAF images of the training set comprise four FAF images spaced over time by a consistent time interval.

16. A method of training a deep learning system comprising:
receiving a plurality of training sets for a plurality of retinas of a plurality of subjects,
wherein each training set of the plurality of training sets comprises training FAF images for at least two different points in time;
generating training input for a deep learning system using the plurality of training sets; and
training the deep learning system to generate a predicted growth output based on FAF image data for a retina of a selected subject, wherein the predicted growth output indicates a predicted growth of a geographic atrophy (GA) lesion in the retina with respect to at least one future point in time.

17. The method of claim 16, wherein the predicted growth output comprises a growth image illustrating an area of the retina predicted to be affected by the GA lesion with respect to a future point in time.

18. The method of claim 16, wherein the predicted growth output comprises a computed area for an entire area of the retina predicted to be affected by the GA lesion with respect to a future point in time.

19. A system comprising:
one or more data processors; and
a non-transitory computer readable storage medium containing instructions which, when executed on the one or more data processors, cause the one or more data processors so that the following steps are executed:
receiving fundus autofluorescence (FAF) image data for a retina of a subject;
wherein the FAF image data includes a first FAF image associated with a first point in time;
generating an image input for a deep learning system using the FAF image data; and
generating, via the deep learning system, a predicted growth output for a geographic atrophy (GA) lesion in the retina using the image input;
wherein the predicted growth output is associated with at least one future point in time after the first point in time.

20. The system of claim 19, wherein the predicted growth output comprises a first growth image that illustrates a first predicted region of growth for the GA lesion with respect to the retina of the subject between a first reference point in time and a first future point in time after the first reference point in time.
